(19) United States
(12) Patent Application Publication
(10) Pub. No.: US 2022/0211283 A1
(43) Pub. Date: Jul. 7, 2022

(54) METHODS FOR BLOOD PRESSURE CALIBRATION SELECTION AND MODELING METHODS THEREOF

(71) Applicant: VITA-COURSE TECHNOLOGIES (HAINAN) CO., LTD., Haikou, Hainan (CN)

(72) Inventors: Jian DENG, Haikou (CN); Chuanmin WEI, Haikou (CN); Jun TAO, Guangzhou (CN); Ziming DENG, Haikou (CN)

(73) Assignee: VITA-COURSE TECHNOLOGIES (HAINAN) CO., LTD., Haikou, Hainan (CN)

(21) Appl. No.: 17/656,416

(22) Filed: Mar. 24, 2022

Related U.S. Application Data
(63) Continuation of application No. PCT/CN2019/107935, filed on Sep. 25, 2019.

(51) Int. Cl. A61B 5/0215; A61B 5/00

(52) U.S. Cl. CPC A61B 5/02156; A61B 5/7246; A61B 5/02154

(57) ABSTRACT

The present disclosure provides a method for blood pressure calibration selection. The method may include inputting a sample set including data files of a plurality of subjects, the data file of each subject including a plurality of sample PPG waveforms and corresponding blood pressure; obtaining calibration data of the each subject in the sample set, the calibration data at least including first calibration data and second calibration data in different blood pressure states; selecting at least one feature parameter of the plurality of sample PPG waveforms; obtaining a value distribution of a feature parameter among the at least one feature parameter in the sample set based on values of the feature parameter in the first calibration data and the second calibration data; and determining calibration data corresponding to a PPG waveform to be detected by comparing the feature parameter of the PPG waveform to be detected with the value distribution.

### CROSS-REFERENCE TO RELATED APPLICATIONS

This application is a Continuation of International Application No. PCT/CN2019/107935, filed on Sep. 25, 2019, the entire contents of which are hereby incorporated by reference.

### TECHNICAL FIELD

The present disclosure generally relates to the field of the medical technology, and more particularly, relates to methods for blood pressure calibration selection and modeling methods thereof.

### BACKGROUND

With the development of mobile medical technology, in addition to traditional invasive and noninvasive measurements of continuous blood pressure measurements, wearable blood pressure measurers based on photoplethysmography (PPG) are increasingly widely used. The invasive measurement is easy to cause damage to blood vessels of subjects, and is accompanied by potential risks, while the traditional noninvasive measurement has great problems in signal stability and signal-to-noise ratio. The PPG has many advantages, such as noninvasive, simple operation, stable performance, etc.

In the PPG, accuracy of algorithms for converting PPG waveforms into pressure waveforms may be improved based on a calibration. The more times of the calibration is, the more enhanmance of the accuracy of the algorithms may be. Therefore, it is desirable to provide methods for blood pressure calibration selection, which can improve the accuracy of the algorithms using multiple calibration data.

### SUMMARY

One aspect of some embodiments of the present disclosure provides a method for blood pressure calibration selection. The method may be implemented by a computer device including at least one processor and at least one storage device. The method may include inputting a sample set. The sample set may include data files of a plurality of subjects. The data file of each subject among the plurality of subjects may include a plurality of sample PPG waveforms and corresponding blood pressure. The method may further include obtaining calibration data of the each subject in the sample set. The calibration data may at least include first calibration data and second calibration data in different blood pressure states. The method may further include selecting at least one feature parameter of the plurality of sample PPG waveforms. The method may further include obtaining a value distribution of a feature parameter among the at least one feature parameter in the sample set based on a plurality of values of the feature parameter in the first calibration data and the second calibration data. The method may further include obtaining a comparison result by comparing the feature parameter of a PPG waveform to be detected with the corresponding value distribution. The method may further include determining calibration data corresponding to the PPG waveform to be detected based on the comparison result.

In some embodiments, the first calibration data may include data in a normal blood pressure state. The first calibration data may be recorded as low calibration data. The second calibration data may include data in a high blood pressure state. The second calibration data may be recorded as high calibration data.

In some embodiments, the low calibration data may be obtained from a first process. The first process may include determining a minimum value of systolic blood pressure of the each subject in the sample set, and determining data corresponding to the minimum value as the low calibration data.

In some embodiments, the high calibration data may be obtained from a second process. The second process may include determining data indicating that a difference between systolic blood pressure of the each subject and the minimum value of the systolic blood pressure of the each subject in the sample set is greater than a threshold A and the systolic blood pressure of the each subject is greater than a threshold B, and determining the data as the high calibration data.

In some embodiments, the threshold A may be 20 millimeters of mercury (mmHg), and the threshold value B may be 130 mmHg.

In some embodiments, the feature parameter among the at least one feature parameter may be determined based on at least one of an original waveform, a first-order derivative waveform, a second-order derivative waveform, a third-order derivative waveform, or a fourth-order derivative waveform of the sample PPG waveform.

In some embodiments, the feature parameter among the at least one feature parameter may include at least one of time amount, area amount, or amplitude amount.

In some embodiments, the obtaining a value distribution of a feature parameter among the at least one feature parameter in the sample set based on a plurality of values of the feature parameter in the first calibration data and the second calibration data may include drawing a two-dimensional (2D) density map and/or a three-dimensional (3D) density map for the feature parameter based on the plurality of values of the feature parameter in the first calibration data and the second calibration data.

In some embodiments, the drawing a 2D density map may include establishing an XY coordinate system, obtaining a plurality of discrete points, each of the plurality of discrete points being obtained by setting a value of the feature parameter in the first calibration data corresponding to the each subject as an X-axis coordinate and setting a value of the feature parameter in the second calibration data corresponding to the each subject as a Y-axis coordinate, and obtaining the 2D density map based on a density distribution of the plurality of discrete points.

In some embodiments, the drawing the 3D density map may include generating a set of correct label data and a set of error label data based on a value of the feature parameter in the first calibration data corresponding to the each subject, a value of the feature parameter in the second calibration data corresponding to the each subject, and a value of the feature parameter in a sample PPG waveform of the each subject other than the calibration data.

In some embodiments, the comparing the feature parameter of a PPG waveform to be detected with the corresponding value distribution may include comparing the feature parameter of the PPG waveform to be detected with the 2D density map and/or the 3D density map. The comparing the feature parameter of the PPG waveform to be detected with the 2D density map and/or the 3D density map may include generating coordinates of at least two points by combining a value of the feature parameter in the PPG waveform to be detected with the values of the feature parameter in the calibration data, and obtaining a relationship between the at least two points and a maximum density point in the 2D density map and/or the 3D density map.

In some embodiments, the comparing the feature parameter of the PPG waveform to be detected with the 2D density map and/or the 3D density map may further include determining a point in the at least two points that is closer to the maximum density point in the 2D density map and/or the 3D density map, and designating calibration data corresponding to the point as the calibration data corresponding to the PPG waveform to be detected.

In some embodiments, the comparing the feature parameter of a PPG waveform to be detected with the corresponding value distribution may include comparing the feature parameter of the PPG waveform to be detected with the 2D density map and/or the 3D density map. The comparing the feature parameter of the PPG waveform to be detected with the 2D density map and/or the 3D density map may include generating coordinates of at least two points by combining a value of the feature parameter in the PPG waveform to be detected with the values of the feature parameter in the calibration data, and obtaining a distance between each of the at least two points and a point obtained from calibration data related to the PPG waveform to be detected.

In some embodiments, the comparing the feature parameter of the PPG waveform to be detected with the 2D density map and/or the 3D density map may further include determining a point in the at least two points that is closer to the point obtained from the calibration data related to the PPG waveform to be detected, and designating calibration data corresponding to the point as the calibration data corresponding to the PPG waveform to be detected.

In some embodiments, an X-axis coordinate and a Y-axis coordinate of the point obtained from the calibration data related to the PPG waveform to be detected may be the values of the feature parameter in the calibration data.

Another aspect of some embodiments of the present disclosure provides a modeling method of a method for blood pressure calibration selection. The method may include inputting a sample set. The sample set may include data files of a plurality of subjects. The data file of each subject of the plurality of subjects may include a plurality of sample photoplethysmography (PPG) waveforms and corresponding blood pressure. The method may further include allocating the sample set into a set of training data and a set of test data. The method may further include obtaining calibration data of the set of test data, recording the calibration data of the set of test data as test calibration data, selecting one of the data in the set of test data other than the test calibration data as the test data, and determining data with a minimum difference between systolic blood pressure in the test calibration data and systolic blood pressure corresponding to the test data as calibration result data of the test data. The method may further include training an initial model based on an input of a sample PPG waveform in the set of training data and an output of corresponding calibration data. The method may further include obtaining an output of a trained model by inputting a sample PPG waveform in the test data, obtaining a comparison result by comparing whether the output of the trained model is consistent with the calibration data, and determining accuracy of the trained model based on the comparison result.

In some embodiments, the method further include obtaining calibrated data of the set of training data, recording the calibrated data of the set of training data as training calibration data, drawing a 2D density map for at least one feature parameter in the sample PPG waveform based on the training calibration data, and obtaining a first set of output results by comparing the at least one feature parameter of the test data with the corresponding 2D density map.

In some embodiments, the method may further include drawing a 3D density map for the at least one feature parameter in the sample PPG waveform based on the training calibration data and obtaining a second set of output results by comparing the at least one feature parameter of the test data with the corresponding 3D density map.

In some embodiments, the method may further include obtaining a final set of final outputs by processing the first set of output results and the second set of output results according to a collective voting algorithm.

Another aspect of some embodiments of the present disclosure provides a system for blood pressure calibration selection. The system may include an input module, a calibration obtaining module, a parameter selection module, a distribution obtaining module, a comparison module, and an output module. The input module may be configured to input a sample set. The sample set may include data files of a plurality of subjects. The data file of each subject among the plurality of subjects may include a plurality of sample photoplethysmography (PPG) waveforms and corresponding blood pressure. The calibration obtaining module may be configured to obtain calibration data of the each subject in the sample set. The calibration data may at least include first calibration data and second calibration data in different blood pressure states. The parameter selection module may be configured to select at least one feature parameter of the plurality of sample PPG waveforms. The distribution obtaining module may be configured to obtain a value distribution of a feature parameter among the at least one feature parameter in the sample set based on a plurality of values of the feature parameter in the first calibration data and the second calibration data. The comparison module may be configured to obtain a comparison result by comparing the feature parameter of a PPG waveform to be detected with the corresponding value distribution. The output module may be configured to determine calibration data corresponding to the PPG waveform to be detected based on the comparison result.

Still another aspect of some embodiments of the present disclosure provides a device for blood pressure calibration selection. The device may include at least one processor and at least one memory. The at least one memory may be configured to store instructions. The at least one processor may be configured to execute at least a portion of the instructions to implement a method for blood pressure calibration selection. The method may include inputting a sample set. The sample set may include data files of a plurality of subjects. The data file of each subject among the plurality of subjects may include a plurality of sample PPG waveforms and corresponding blood pressure. The method may further include obtaining calibration data of the each subject in the sample set. The calibration data may at least include first calibration data and second calibration data in different blood pressure states. The method may further include selecting at least one feature parameter of the plurality of sample PPG waveforms. The method may further include obtaining a value distribution of a feature parameter among the at least one feature parameter in the sample set based on a plurality of values of the feature parameter in the first calibration data and the second calibration data. The method may further include obtaining a comparison result by comparing the feature parameter of a PPG waveform to be detected with the corresponding value distribution. The method may further include determining calibration data corresponding to the PPG waveform to be detected based on the comparison result.

Still another aspect of some embodiments of the present disclosure provides a computer readable storage medium. The storage medium may be configured to store instructions. When the instructions are executed by at least one processor, at least a portion of the instructions may direct the at least one processor to perform a method for blood pressure calibration selection. The method may include inputting a sample set. The sample set may include data files of a plurality of subjects. The data file of each subject among the plurality of subjects may include a plurality of sample PPG waveforms and corresponding blood pressure. The method may further include obtaining calibration data of the each subject in the sample set. The calibration data may at least include first calibration data and second calibration data in different blood pressure states. The method may further include selecting at least one feature parameter of the plurality of sample PPG waveforms. The method may further include obtaining a value distribution of a feature parameter among the at least one feature parameter in the sample set based on a plurality of values of the feature parameter in the first calibration data and the second calibration data. The method may further include obtaining a comparison result by comparing the feature parameter of a PPG waveform to be detected with the corresponding value distribution. The method may further include determining calibration data corresponding to the PPG waveform to be detected based on the comparison result.

### BRIEF DESCRIPTION OF THE DRAWINGS

The present disclosure is further described in terms of exemplary embodiments. These exemplary embodiments are described in detail with reference to the drawings. These embodiments are non-limiting exemplary embodiments, in which like reference numerals represent similar structures throughout the several views of the drawings, and wherein:

FIG. 1 is a block diagram illustrating an exemplary system for blood pressure calibration selection according to some embodiments of the present disclosure;

FIG. 2 is a flowchart illustrating an exemplary process for blood pressure calibration selection according to some embodiments of the present disclosure;

FIG. 3 is a flowchart illustrating an exemplary process for preprocessing according to some embodiments of the present disclosure;

FIG. 4 is a flowchart illustrating an exemplary process for drawing a 2D density map and/or a 3D density map according to some embodiments of the present disclosure;

FIG. 5 is a flowchart illustrating an exemplary process for determining calibration data corresponding to a PPG waveform to be detected according to some embodiments of the present disclosure;

FIG. 6 is a density map illustrating an exemplary process for placing feature parameters into a 2D density map for comparison according to some embodiments of the present disclosure; and

FIG. 7 is a flowchart illustrating an exemplary process for modeling according to some embodiments of the present disclosure.

### DETAILED DESCRIPTION

In order to illustrate the technical solutions related to the embodiments of the present disclosure, brief introduction of the drawings referred to in the description of the embodiments is provided below. Obviously, drawings described below are only some examples or embodiments of the present disclosure. Those having ordinary skills in the art, without further creative efforts, may apply the present disclosure to other similar scenarios according to these drawings. Unless obviously obtained from the context or the context illustrates otherwise, the same numeral in the drawings refers to the same structure or operation.

As used in the disclosure and the appended claims, the singular forms “a”, “an”, and “the” include plural referents unless the content clearly dictates otherwise. In general, the terms “comprise”, “comprises”, and/or “comprising”, “include”, “includes”, and/or “including” merely prompt to include steps and elements that have been clearly identified, and these steps and elements do not constitute an exclusive listing. The methods or devices may also include other steps or elements.

Although the present disclosure makes various references to some modules in the system according to some embodiments of the present disclosure, any number of different modules can be used and run on the client and/or server. The modules are merely illustrative, and different modules may be used in different aspects of the systems and methods.

The flowcharts used in the present disclosure illustrate operations that systems implement according to some embodiments of the present disclosure. It should be understood that the operations of the flowcharts may be implemented not in order. Conversely, the operations may be implemented in inverted order, or simultaneously. Moreover, one or more other operations may be added to the flowcharts or one or more operations may be removed from the flowcharts.

The present disclosure relates to a method for blood pressure calibration selection and a modeling method thereof. According to some embodiments of the present disclosure, the method may include extracting a plurality of feature parameters based on a plurality of sample PPG waveforms provided by a plurality of subjects, and drawing a density map for one or more feature parameters among the plurality of feature parameters to display corresponding relationships between PPG waveforms and blood pressure of most subjects in different blood pressure states. The method may include comparing a corresponding relationship between a PPG waveform and blood pressure of a subject in a blood pressure state with the corresponding relationships (the density map) of the most subjects to assist in determining a blood pressure state of the subject at this time, thereby selecting appropriate calibration for accurately measuring blood pressure of the subject. For example, 3-4 sample PPG waveforms may be collected for each subject, and measurement results of the plurality of subjects may be used as a reference, thereby eliminating the need to collect too many samples from a same subject. The method may be applied to many fields, for example, guardianship (including elderly guardianship, middle-aged guardianship, youth guardianship, children guardianship, or the like, or any combination thereof), medical diagnosis (including ECG diagnosis, pulse diagnosis, blood pressure diagnosis, blood oxygen diagnosis, or the like, or any combination thereof), motion monitoring (including long-distance running, middle and/or short distance running, sprinting, cycling, rowing, archery, horse riding, swimming, mountain climbing, or the like, or any combination thereof), hospital nursing (including severe patient monitoring, genetic disease patient monitoring, emergency patient monitoring, or the like, or any combination thereof), pet nursing (critical pet nursing, newborn pet nursing, home pet nursing, or the like, or any combination thereof), or the like, or any combination thereof.

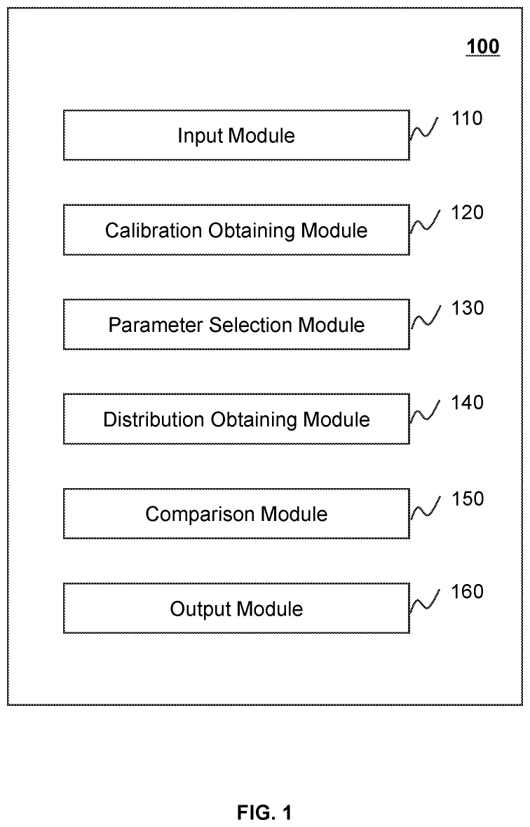

FIG. 1 is a block diagram illustrating an exemplary system 100 for blood pressure calibration selection according to some embodiments of the present disclosure. In some embodiments, the system 100 for blood pressure calibration selection may be provided. The system 100 may include an input module 110, a calibration obtaining module 120, a parameter selection module 130, a distribution obtaining module 140, a comparison module 150, and an output module 160. In some embodiments, the input module 110 may be configured to input a sample set. The sample set may include a plurality of data files of a plurality of subject. The data file of each subject among the plurality of subjects may include a plurality of sample PPG waveforms and corresponding blood pressure. In some embodiments, the calibration obtaining module 120 may be configured to obtain calibration data of the each subject in the sample set. The calibration data may at least include first calibration data and second calibration data in different blood pressure states. In some embodiments, the parameter selection module 13 may be configured to select at least one feature parameter of the plurality of sample PPG waveforms. In some embodiments, the distribution obtaining module 140 may be configured to obtain a value distribution of a feature parameter among the at least one feature parameter in the sample set based on a plurality of values of the feature parameter in the first calibration data and the second calibration data. In some embodiments, the comparison module 150 may be configured to obtain a comparison result by comparing the feature parameter of a PPG waveform to be detected and the corresponding value distribution. In some embodiments, the output module 160 may be configured to determine calibration data corresponding to the PPG waveform to be detected based on the comparison result.

It should be understood that the system 100 and the modules 110-160 thereof shown in FIG. 1 may be implemented in various ways. For example, in some embodiments, the system 100 and the modules 110-160 thereof may be implemented by a hardware, a software, or a combination thereof. The hardware may be implemented by a dedicated logic. The software may be stored in a storage device which may be executed by an appropriate instruction execution system, such as a microprocessor or dedicated design hardware. Those skilled in the art may understand that the above system may be implemented using computer-executable instructions and/or included in processor control codes. For example, carrier media (e.g., a disk, a CD, a DVD-ROM, etc.), programmable memories (e.g., a read-only memory (firmware)), or data carriers (e.g., an optical carrier or an electronic signal carrier) may provide the codes. The system 100 and the modules 110-160 thereof of the present disclosure may not only be implemented by a very large-scale integrated (VLSI) circuit or a gate array, a semiconductor such as a logic chip, a transistor, etc., a hardware circuit of a programmable hardware device such as a field programmable gate array, a programmable logic device, etc., may also be implemented by software executed by various types of processors. Alternatively, the system 100 and the modules 110-160 thereof may also be implemented by a combination of the above hardware circuits and software (e.g., firmware).

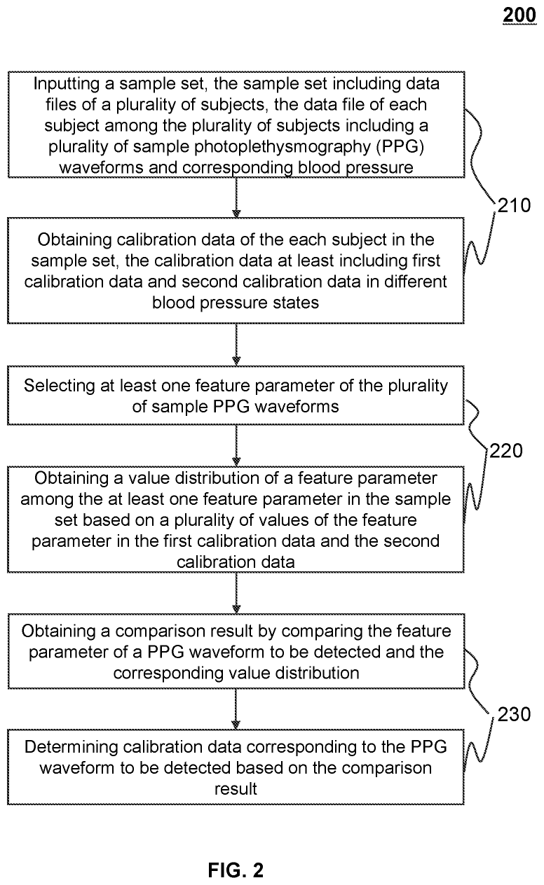

In some embodiments of the present disclosure, a method for blood pressure calibration selection may be provided. FIG. 2 is a flowchart illustrating an exemplary process for blood pressure calibration selection according to some embodiments of the present disclosure.

In some embodiments, the process for blood pressure calibration selection may include the following operations.

In 210, preprocessing may be performed. The preprocessing may include inputting a sample set and obtaining calibration data based on the sample set.

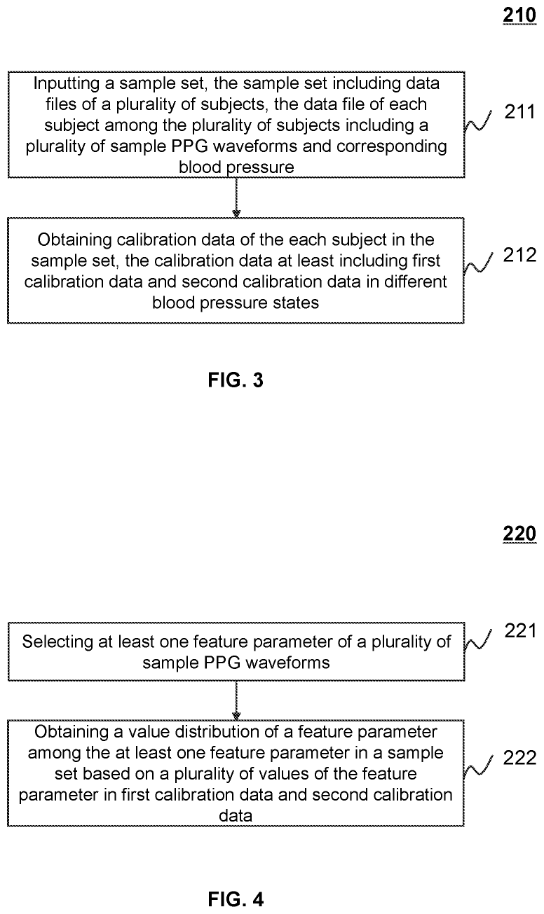

FIG. 3 is a flowchart illustrating an exemplary process for preprocessing according to some embodiments of the present disclosure.

In 211, a sample set may be input. The sample set may include data files of a plurality of subjects. The data file of each subject among the plurality of subjects may include a plurality of sample PPG waveforms and corresponding blood pressure. In some embodiments, the operation 211 may be executed by the input module 110.

In some embodiments, the sample PPG waveform may include a waveform obtained based on PPG. The PPG refers to a non-invasive detection manner that detects changes of blood volume in living tissue through a photoelectric means. The PPG may obtain the blood pressure (including systolic and diastolic blood pressure) of the subject by extracting a PPG waveform using a PPG measurement device on a specific body portion (e.g., a fingertip, an ear, a forehead, a nose, etc.) of the subject and converting the obtained PPG waveform into a pressure waveform based on a certain algorithm. In order to improve calculation accuracy of converting the PPG waveform into the pressure waveform, a corresponding relationship between the PPG waveform and the blood pressure may be determined using a calibration. The calibration may include measuring the blood pressure of the subject using a standard blood pressure measurement device (e.g., a mercury sphygmomanometer) when using the PPG measurement device to measure the PPG waveform of the subject or about one minute before and/or after the time. The solution of the present disclosure may be provided on the premise that a plurality of calibration data of the plurality of subjects have been obtained, and the corresponding relationships between the PPG waveforms and the pressure waveform of most subjects have a certain correlation.

In 212, calibration data of the each subject in the sample set may be obtained. The calibration data may at least include first calibration data and second calibration data in different blood pressure states. In some embodiments, the operation 212 may be executed by the calibration obtaining module 120.

In some embodiments, the different blood pressure states may include a normal blood pressure state, and a high blood pressure state. The high blood pressure state refers to a blood pressure state with a large change in the blood pressure relative to the normal blood pressure (e.g., the blood pressure increased by 20 mmHg or more). When the subject is in the normal blood pressure state, one calibration may be performed. Relevant data obtained at this time may be first calibration data, which is recorded as low calibration data. The relevant data may at least include a PPG waveform and corresponding blood pressure. When the blood pressure is increased by 20 mmHg or more, another calibration may be performed. Relevant data obtained at this time may be second calibration data, which is recorded as high calibration data. The blood pressure corresponding to the another calibration may indicate a blood pressure range of the subject in the high blood pressure state.

In some embodiments, a plurality of PPG waveforms of the subjects and corresponding blood pressure may be extracted in the sample set, wherein the corresponding blood pressure may be obtained by the standard blood pressure measurement device. The low calibration data and the high calibration data may be determined based on a range of systolic blood pressure. For example, when the systolic blood pressure is minimum, the PPG waveform and relevant data corresponding to the systolic blood pressure may be designated as the low calibration data of the subject. When the systolic blood pressure value is maximum, the PPG waveform and relevant data corresponding to the systolic blood pressure may be designated as the high calibration data of the subject. In some embodiments, the high calibration data may also be obtained in another way. For example, a PPG waveform and relevant data that the systolic blood pressure of the subject is greater than 130 mmHg and a difference between the systolic blood pressure of the subject and a minimum value of the systolic blood pressure of the subject is greater than 20 mmHg may be determined as the high calibration data. In some embodiments, if there are at least two pieces of data that the systolic blood pressure is greater than 130 mmHg and the difference between the systolic blood pressure and the minimum value of the systolic blood pressure is greater than 20 mmHg, one piece of data may be randomly selected from the at least two pieces of of data that meet the requirements, and designated as the high calibration data.

In some embodiments, in addition to the high calibration data and the low calibration data, calibration data in a variety of other blood pressure states between the high calibration data and the low calibration data may be selected. For example, after the low calibration data is designated, one piece of calibration data may be added when the systolic blood pressure is increased by every 20 mmHg. When a difference between the systolic blood pressure and the systolic blood pressure of the high calibration data is less than 20 mmHg, the calibration data may not be added when a difference between the systolic blood pressure and the systolic blood pressure of the high calibration data is less than 20 mmHg. As another example, when the subject is in the normal blood pressure state, the calibrated data may be designated as first calibration data. When the blood pressure is increased by 20 mmHg, corresponding data at this time may be designated as second calibration data. When the blood pressure continues to be increased by 20 mmHg, corresponding data at this time may be designated as third calibration data. By analogy, more than two pieces of calibration data may be obtained, thereby improving accuracy of the blood pressure.

In 220, a 2D density map and/or a 3D density map may be drawn. In some embodiments, the operation 220 may include operation 221 and operation 222 illustrated in FIG. 4.

FIG. 4 is a flowchart illustrating an exemplary process 400 for drawing a 2D density map and/or a 3D density map according to some embodiments of the present disclosure.

In 221, at least one feature parameter of a sample PPG waveform may be selected. In some embodiments, the operation 221 may be executed by the parameter selection module 130.

In some embodiments, the at least one feature parameter may be extracted from the sample PPG waveform. In some embodiments, the at least one feature parameter may be determined based on an original waveform, a first-order derivative waveform, a second-order derivative waveform, a third-order derivative waveform, a four-order derivative waveforms, or the like, or any combination thereof, of the sample PPG waveform. In some embodiments, the at least one feature parameter may include time amount, area amount, amplitude amount, or the like, or any combination thereof. For example, the at least one feature parameter may include a duration from a trough of the sample PPG waveform to a maximum rising edge of the sample PPG waveform (the maximum rising edge represents a maximum slope of a rising curve), an amplitude at a peak of the sample PPG waveform, a relative ratio of the time, the area, and the amplitude, a ratio of an amplitude at the maximum rising edge of the trough of the sample PPG waveform to the amplitude at the peak of the sample PPG waveform, or the like, or any combination thereof. The amplitude amount refers to an amount reflecting a product of the time and the amplitude. For example, the amplitude amount that is obtained by multiplying the amplitude by the time of the amplitude may reflect the amplitude amount and the time amount at the same time. Based on the above manner, a plurality of feature parameters fi (i=1, 2, 3, . . . , n) of the sample PPG waveform may be extracted.

In 222, a value distribution of a feature parameter among the at least one feature parameter in the sample set may be obtained based on a plurality of values of the feature parameter in first calibration data and second calibration data. In some embodiments, the operation 222 may be executed by the distribution obtaining module 140.

In some embodiments, the 2D density map and/or the 3D density map for the feature parameter may be drawn based on the plurality of values of the feature parameter in the first calibration data and the second calibration data.

In some embodiments, drawing the 2D density map may include establishing an XY coordinate system, obtaining a plurality of discrete points, each of the plurality of discrete points being obtained by setting a value of the feature parameter in the first calibration data corresponding to the each subject as an X-axis coordinate and setting a value of the feature parameter in the second calibration data corresponding to the each subject as a Y-axis coordinate, and obtaining the 2D density map based on a density distribution of the plurality of discrete points.

In some embodiments, drawing the 3D density map may include generating a set of correct label data and a set of error label data based on a value of the feature parameter in the first calibration data corresponding to the each subject, a value of the feature parameter in the second calibration data corresponding to the each subject, and a value of the feature parameter in a sample PPG waveform of the each subject other than the calibration data.

In 230, a comparison result may be obtained by comparing the feature parameter of a PPG waveform to be detected and the corresponding value distribution. In some embodiments, the operation may be executed by the comparison module 150. Calibration data corresponding to the PPG waveform to be detected may be determined based on the comparison result. In some embodiments, the operation may be performed by the output module 160.

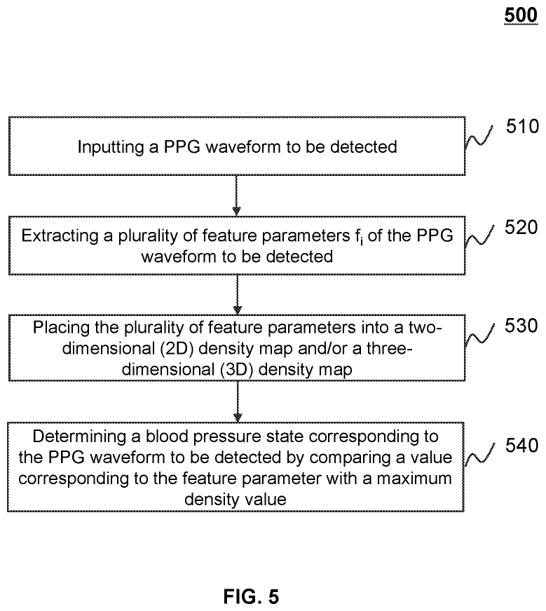

FIG. 5 is a flowchart illustrating an exemplary process for determining calibration data corresponding to a PPG waveform to be detected according to some embodiments of the present disclosure.

In 510, a PPG waveform to be detected may be input.

In 520, a plurality of feature parameters fi (i=1, 2, 3, . . . , n) of the PPG waveform to be detected may be extracted.

In 530, the plurality of feature parameters may be placed into a 2D density map and/or a 3D density map.

In 540, a blood pressure state corresponding to the PPG waveform to be detected may be determined by comparing a value corresponding to the feature parameter with a maximum density value.

In some embodiments, placing the plurality of feature parameters into the 2D density map may include obtaining coordinates of points X and Y by combining the value of the feature parameter in the PPG waveform to be detected with a plurality of values of the feature parameter in calibration data (e.g., the first calibration data, the second calibration data), respectively, and comparing a distance between each of the point X and the point Y and a point with the maximum density value in the 2D density map. For one PPG waveform to be detected, if the PPG waveform to be detected corresponds to a high blood pressure state, coordinates (fi, f_hi_cali) of the point X may be obtained, and the point X may be placed into the 2D density map corresponding to the feature parameter. As used herein, a horizontal coordinate of the point X may refer to a true value of the feature parameter of the PPG waveform to be detected, which is recorded as fi; and a vertical coordinate of the point X may refer to a value corresponding to the feature parameter in high calibration data (i.e., the second calibration data) of the subject corresponding to the PPG waveform to be detected, which is recorded as f_hi_cali. If the PPG waveform to be detected corresponds to a normal blood pressure state, coordinates (f_low_cali, fi) of the point Y may be obtained, and the point Y may be placed into the 2D density map corresponding to the feature parameter. As used herein, a horizontal coordinate of the point Y may refer to a value corresponding to the feature parameter in low calibration data (i.e., the first calibration data) of the subject corresponding to the PPG waveform to be detected, which is recorded as f_low_cali; and a vertical coordinate of the point X may refer to the value fi of the feature parameter of the PPG waveform to be detected. The blood pressure state corresponding to the PPG waveform to be detected may be determined by comparing each of the point X and the point Y with the point corresponding to the maximum density value. For example, if the value (f_low_cali) of the feature parameter is 10 in the low calibration data obtained from the operation 212, the value (f_hi_cali) of the feature parameter is 50 in the high calibration data obtained from the operation 212, and the value (fi) of the feature parameter is 20 in the PPG waveform to be detected, the coordinates of the point X may be determined to be (20, 50), and the coordinates of the point Y may be determined to be (10, 20). The points X and Y may be compared with the point corresponding to the maximum density value in the 2D density map by placing the points X and Y into the 2D density map obtained in the operation 222. If the point X is closer to the point corresponding to the maximum density value than the point Y, the blood pressure state corresponding to the PPG waveform D to be detected may be high blood pressure state. If the point Y is closer to the point corresponding to the maximum density value than the point X, the blood pressure state corresponding to the PPG waveform D to be detected may be the normal blood pressure state. If a distance between the point X and the point corresponding to the maximum density value is the same as a distance between the point Y and the point corresponding to the maximum density value, the blood pressure state corresponding to the PPG waveform D may be determined as the high blood pressure state or the normal blood pressure state.

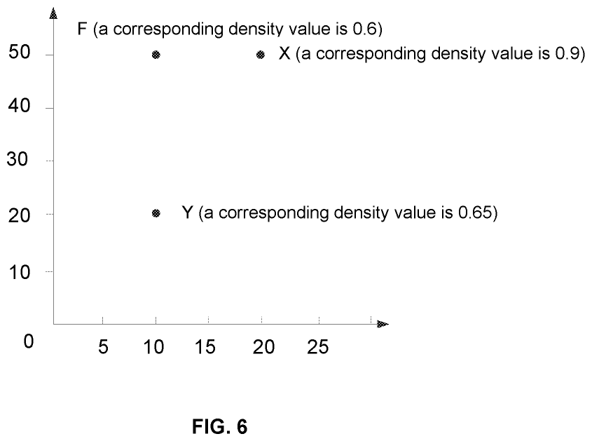

FIG. 6 is a density map illustrating an exemplary process for placing feature parameters into a 2D density map for comparison according to some embodiments of the present disclosure. In the above embodiment, a density value corresponding to the point X (20, 50) may be 0.9 in the 2D density map, a density value corresponding to the point Y (10, 20) may be 0.65 in the 2D density map, and the maximum density value may be 1 after normalization. Therefore, the distance between the point X and the point corresponding to the maximum density value may be closer the distance between the point Y and the point corresponding to the maximum density value, the blood pressure state corresponding to the PPG waveform to be detected may be the high blood pressure state.

In some other embodiments, coordinates of at least two points may be generated by combining the plurality of values of the feature parameter in the PPG waveform to be detected with the plurality of values of the feature parameter in the calibration data, respectively, and a distance may be obtained between each of the at least two points and a point obtained from calibration data related to the PPG waveform to be detected. In the embodiment, a point which is closer to the point obtained from the calibration data related to the PPG waveform to be detected may be determined among the at least two points, and calibration data corresponding to the point may be designated as the calibration data corresponding to the PPG waveform to be detected. An X-axis coordinate and a Y-axis coordinate of the point obtained from the calibration data related to the PPG waveform to be detected may be the values of the feature parameter in the calibrated data. In the embodiment, the points X and Y may be compared with a point formed by the low calibration data and the high calibration data of the subject (the same subject) in the density map, so as to determine the blood pressure state corresponding to the PPG waveform D to be detected. As shown in the above embodiments, the value (f_low_cali) of the feature parameter may be 10 in the low calibration data, the value (f_hi_cali) of the feature parameter may be 50 in the high calibration data, and the value (fi) of the feature parameter may be 20 in the PPG waveform D to be detected. The coordinates of the point X may be (20, 50), the coordinates of the point Y may be (10, 20), and coordinates of a point F formed by the low calibration data and the high calibration data of the subject (the same subject) may be (10, 50). The points X and Y may be placed into the 2D density map obtained from the operation 222. If a distance between the point X and the point F is closer than a distance between the point Y and the point F, the blood pressure state corresponding to the PPG waveform D to be detected may be the high blood pressure state. If the distance between the point Y and the point F is closer than the distance between the point X and the point F, the blood pressure state corresponding to the PPG waveform D to be detected may be the normal blood pressure state. Referring to FIG. 6, a density value corresponding to the point X (20, 50) may be 0.9 in the density map, a density value corresponding to the point Y (10, 20) may be 0.65 in the density map, and a density value corresponding to the point F (10, 50) may be 0.6 in the density map. Therefore, the distance between the point Y and the point F may be closer than the distance between the point X and the point F, the blood pressure state corresponding to the PPG waveform D to be detected may be the normal blood pressure state.

In the above two embodiments, the meaning of comparing with the maximum density value in the 2D density map may include that relevant data of all subjects in the sample set is taken as a reference for the PPG waveform to be detected, which is suitable for a case where little waveforms are collected for the subject. For example, 3 PPG waveforms may be collected for each subject. The meaning of comparing the points formed by the high calibration data and the low calibration data of the subject in the 2D density map may include that when a count (or number) of PPG waveforms collected by the subject is large enough, for example, more than 100, the PPG waveform to be detected may be compared with an actual situation of the subject, which improves accuracy of the obtained data.

In some embodiments, the density map may be obtained according to a kernel density estimation. The kernel density estimate may be a density function used to estimate a location in probability theory, which belongs to one of non-parametric test techniques.

It should be noted that the above descriptions of the processes 200, 210, 220, and 500 are merely for example and illustration, and not intended to limit the scope of disclosure of the present disclosure. For those skilled in the art, various variations and modifications may be made to processes 200, 210, 220, and 500 under the teaching of the present disclosure. However, those variations and modifications may be within the scope of the protection of one or more embodiments of the present disclosure. For example, in 212, the calibration data of the subject may be not limited to be divided into the low calibration and the high calibration data, but also may be divided in other manners.

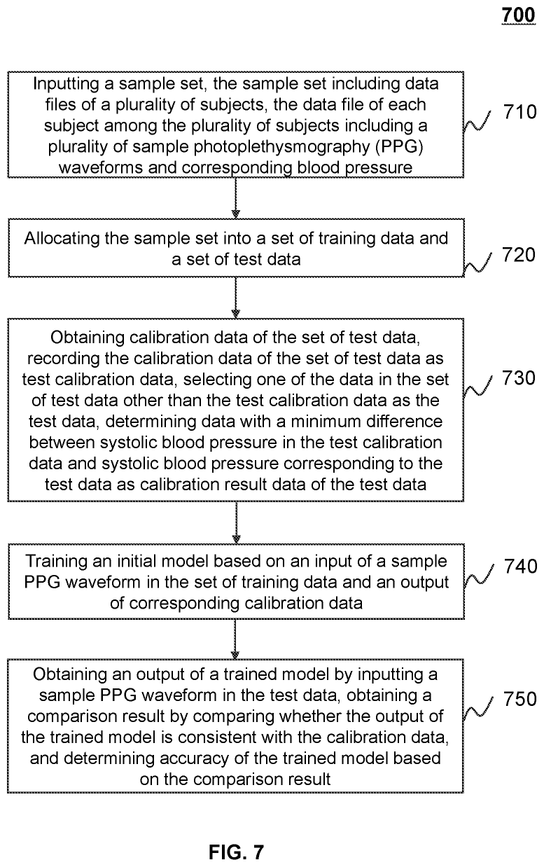

In some embodiments of the present disclosure, a modeling method of a method for blood pressure calibration selection may be provided. FIG. 7 is a flowchart illustrating an exemplary process for modeling according to some embodiments of the present disclosure.

In 710, a sample set may be input. The sample set may include data files of a plurality of subjects. The data file of each subject of the plurality of subjects may include a plurality of sample PPG waveforms and corresponding blood pressure. For example, the data file of the each subject may at least include 3 sample PPG waveforms and corresponding blood pressure.

In 720, the sample set may be allocated into a set of training data and a set of test data. For example, the sample set may be allocated into a set of training data and a set of test data in a ratio of 7:3. The data for the each subject may be allocated into a set of single data. That is, the subjects in the set of training data and the set of test data do not overlap.

In 730, calibration data of the set of test data may be obtained. The calibration data of the set of test data may be recorded as test calibration data. One of the data in the set of test data other than the test calibration data may be designated as the test data. Data with a minimum difference between systolic blood pressure in the test calibration data and systolic blood pressure corresponding to the test data may be determined as calibration result data of the test data.

In 740, an initial model may be trained based on an input of a sample PPG waveform in the set of training data and an output of corresponding calibration data.

In 750, an output of a trained model may be obtained by inputting a sample PPG waveform in the test data, a comparison result may be obtained by comparing whether the output of the trained model is consistent with the calibration data, and accuracy of the trained model may be determined based on the comparison result.

In some embodiments, the modeling method of the method for blood pressure calibration selection may be performed based on a large amount of the obtained data (including PPG waveforms, feature parameters, corresponding blood pressure, and other relevant data) of the subject. The relevant data may include basic information (e.g., account number, a gender, an age, a height, a weight, etc.) of the subject and feature parameters (e.g., time parameters, amplitude parameters, area parameters, etc., obtained during a detection of the PPG waveform) generated based on signal processing.

In some embodiments, all data of the subjects may be classified according to the subjects. Each subject may correspond to a folder. Each folder may include at least three pieces of measurement data, two of which may be determined as calibrated data and one of which may be determined as test data. The data of the subjects may be randomly allocated to the set of training data and the set of test data in a ratio of 7:3 (or 8:2) according to a count (or number) of the folders, which may be recorded as train_data and test_data, respectively.

In some embodiments, low calibration data and high calibration data of each folder may be determined in the set of training data train_data. The low calibration data and the high calibration data in each folder may be determined as the calibration data of the training data, which is recorded as cali.train. The determining the low calibration data and the high calibration data may include selecting data corresponding to a minimum value of systolic blood pressure as the low calibration data based on measured systolic blood pressure, the data being recorded as Calihigh=0; and randomly selecting one of the data as the high calibration data among the data that a value of the systolic blood pressure is greater than a threshold B and a difference between the blood pressure and the minimum value of the systolic blood pressure is greater than a threshold A, the selected data being recorded as Calihigh=1.

In some embodiments, low calibration data and high calibration data of each folder in the set of test data test_data may be determined. The low calibration data and the high calibration data in each folder may be determined as the calibration data of the test data, which is recorded as cali.test. The determining the low calibration data and the high calibration data in the set of test data may be the same as the determining the low calibration data and high calibration data in the set of training data. The low calibration data may be recorded as Calihigh=0, and the high calibration data may be recorded as Calihigh=1. In some embodiments, one piece of data may be randomly designated after removing the low calibration data and the high calibration data from the set of test data of each folder. The piece of data may be recorded as data.test_sampled. The calibration Calihigh of the data corresponding to a minimum difference between the corresponding systolic blood pressure and the systolic blood pressure of low calibration/high calibration may be designated as the calibration result data of the test data, which is recorded as test_ind.

In some embodiments, one or more variables with representativeness among the feature parameters may be selected to form a set of feature parameter cornames5. For each feature parameter in cornames5, a value feature0 of the feature parameter may be obtained from the low calibration data cali.train0 of the set of training data, a value feature1 of the feature parameter may be obtained from the high calibration data cali.train1 of the set of training data, a value feature0.test of the feature parameter feature0.test may be obtained from the low calibration data of the set of test data, and a value feature1.test of the feature parameter may be obtained from the high calibration data cali.train0 of the set of test data. In some embodiments, a 2D density map may be drawn for each feature parameter in the set of feature parameters based on the feature0, feature1, feature0.test, and feature1.test.

In some embodiments, in the 2D density map, a horizontal coordinate and a vertical coordinate corresponding to different N values (e.g., N=7, 9, 13, 17, 21, 25, 31) may be a value feature0 corresponding to the low calibration data and a value feature1 corresponding to the high calibration data of the set of training data, respectively. In some embodiments, a normalization may be performed using a Z-axis. That is, a dimensional expression may be transformed into a dimensionless expression to be a scalar with a value between 0 and 1, which is convenient for comparison and processing in subsequent operations.

Whether the test data data.test_sampled is the low calibration data or the high calibration data may be determined based on a distance between the test data and a point (feature0, feature1). The distance may be recorded as feature_ind2. In some embodiments, a correlation coefficient of the feature_ind2 and test_ind corresponding to each N value of each feature parameter may be recorded as fea_cor_2D. The fea_cor_2 D refers to a m×n matrix, wherein n refers to a value of N, and m refers to a count (or number) of feature parameters in the set of feature parameters cornames5. In some embodiments, the test_ind may be the calibration result data, a correlation coefficient between the feature_ind2 and the test_ind corresponding to each N value of each feature parameter may be recorded as the fea_cor_2D, an optimal ranking of N value may be determined by comparing the correlation coefficient fea_cor_2D with the calibration result data so as to select an optimal N value or a suboptimal N value in the calculation process to improve the accuracy of the obtained result.

In the above embodiments, a first set of output results may be obtained. That is, the first set of output results may be obtained from the 2D density map.

In some embodiments, the selecting the feature parameters in the set of feature parameter cornames5 may include selecting the one or more variables with representativeness among the feature parameters, wherein the representativeness represents a correlation between the feature parameter and the blood pressure. The stronger the representativeness is, the higher the correlation between the feature parameter and blood pressure may be. That is, a change of the variable may change synchronously with the blood pressure. In some embodiments, the selecting the one or more variables with representativeness may include determining the correlation between the variable and the blood pressure, for example, selecting a variable with a correlation greater than 0.2 or 0.3.

In some embodiments, the N value may represent a granularity. The N value may be used to draw the density map. That is, the N value may represent an interval between a horizontal axis and a vertical axis when drawing the density map. The smaller the N value is, the coarser a division of the density map may be, the more data each block of the map may be, and the wider a range of the each block may be. The greater the N value is, the finer the division of the density map may be, the less data in the each block of the map may be, and the smaller the range of each block may be. Therefore, an appropriate N value may be selected to draw an accurate and fine density map. The N value with a best correlation may be selected by ranking the N values.

In some embodiments, low calibration data and high calibration data may be selected and recorded as Calihigh=0/1 for each folder in the set of training data train_data, respectively, and then remaining data after removing the low calibration data and the high calibration data may be determined as training samples and recorded as train_sampled.data. The training samples may include a plurality of folders. Each of the plurality of folders may correspond to a subject, and each of the plurality of folders may include a plurality of waveform/blood pressure data, representing a PPG waveform of the subject and corresponding blood pressure. In some embodiments, an i-th feature parameter may be selected in the set of feature parameters cornames5. For a k-th folder in the training sample, a value corresponding to the low calibration data may be recorded as f0, a value corresponding to the high calibration data may be recorded as f1, and a value corresponding to one of the remaining data may be recorded as c. For an indicator, if the data corresponding to c is in the normal blood pressure state, it may be determined that l(c, f1)=0, and l(f0, c)=1. As used herein, the value “1” may represent correct, the value “0” may represent error, l(f0, c)=1 may represent that c corresponding to the low calibration data is correct, and l(c, f1)=0 may represent that c corresponding to the high calibration data is error. Correspondingly, if the data corresponding to c is in the high blood pressure state, it may be determined that l(c, f1)=1, and l(f0, c)=0. In some embodiments, two sets of data (c, f1, l(c, f1), k) and (f0, c, i(f0, c), k) may be generated for the above data. The two sets may record a set (l=1) of points that a label of the blood pressure state is correct and a set (l=2) of points of that the label of the blood pressure state is error in the training samples.

In some embodiments, low calibration data and high calibration data may be selected and recorded as Calihigh=0/1 for each folder in the set of training data train_data, respectively, and remaining data after removing the low calibration data and the high calibration data may be determined as test samples and recorded as test_sampled.data for each folder in the set of test data. For each feature parameter and each piece of data in the test_sampled.data, a selection of the high calibration data and the low calibration data may be determined, and an obtained result may be recorded as feature_ind3. In some embodiments, the determination may include, for each feature parameter in the selected feature parameter set cornames5, recording a value corresponding to the low calibration data of the k-th folder in the test samples as F0, recording a value corresponding to the high calibration data of the k-th folder in the test samples as F1, and recording a value corresponding to one piece of of the remaining data of the k-th folder in the test samples as C. For each folder in the train.data, (feature0.folder, feature1.folder) may be recorded as the value corresponding to the low calibration data and the value corresponding to the high calibration data of one feature parameter in the folder. If a distance between the (feature0.folder, feature1.folder) and (F0, F1) is less than a distance parameter rad, the points of the folder in the 3D density map may be included in ensemble. If a distance between (F0, C, 1) and a point in the ensemble and/or a distance between (C, F1, 0) and the point in the ensemble are less than a distance between (F0, C, 0) and the point in the ensemble and/or a distance between (C, F1, 1) and the point in the ensemble, feature_ind3 may be determined to be 1. If the distance between (F0, C, 1) and a point in the ensemble and/or the distance between (C, F1, 0) and the point in the ensemble are greater than or equal to the distance between (F0, C, 0) and the point in the ensemble and/or the distance between (C, F1, 1) and the point in the ensemble, the feature_ind3 may be determined to be 0.

In the above embodiments, a second set of output results may be obtained. That is, the second set of output results may be obtained from the 3D density map.

In some embodiments, the points included in the ensemble may be used as reference data, which may avoid a case that data in some special situations affects the accuracy of the calculation. In other words, the folder in the train.data may be filtered using the above operation. If the distance between (feature0.folder, feature1. folder) and (F0, F1) is less than the distance parameter rad, the folder may be related to the folder corresponding to the test data and may be used as reference data. If the distance between (feature0.folder, feature1. folder) and (F0, F1) is greater than the distance parameter rad, the folder may not be appropriate to be used as the reference data, and the folder need to be removed in the 3D density map, thereby further improving the accuracy.

In some embodiments, the distance parameter rad may be determined based on Equation (1):

$$rad = min ( \frac{max ( feature 1 ) - min ( feature 1 )}{N} , \frac{max ( feature 0 ) - min ( feature 0 )}{N} ) ; \qquad ( 1 )$$

where the distance parameter rad may represent a smaller value of a length or a width of each actual physical block in the density map.

In some embodiments, for the first set of output results and the second set of output results, a final output result feature_ind may be obtained by comparing the feature_ind2 and the feature_ind3 based on a collective voting algorithm (also referred to as a majority voting algorithm). In an array including n non-negative elements, an element that a count (or number) of times of occurrences is greater than n/2 may be output based on the majority voting algorithm. The output result may be obtained according to a process. The process may include scanning the entire array; saving each number exited in the array into a count in a table, the count indicating the times of occurrences; scanning all counts and comparing the counts with n/2; and outputting a number corresponding to the count if the count is greater than n/2.

In some embodiments, the results obtained from the 2D density map and the results obtained from the 3D density map may be compared based on the collective voting algorithm, and the results with the most times of occurrences may be selected from the first set of output results and the second set of output results, which may avoid errors and improve the accuracy of the results.

It should be noted that both the first set of output results and the second set of output results may be used as the final output results. That is, the first set of output results and/or the second set of output results may be directly used as the final output results without comparison. However, the comparison may improve the accuracy of the results.

It should be noted that the above description is merely for the convenience of description, but not intended to limit the present disclosure to the scope of the embodiments. For persons having ordinary skills in the art, multiple variations or modifications may be made under the teachings of the present disclosure. However, those variations and modifications do not depart from the scope of the present disclosure.

In some embodiments of the present disclosure, a device for blood pressure calibration selection may be provided. In some embodiments, the device may include at least one processor and at least one memory, the at least one memory may be configured to store instructions, the at least one processor may be configured to execute at least a portion of the instructions to implement the operations as described above.

In some embodiments of the present disclosure, a computer readable storage medium may be provided. In some embodiments, the storage medium may be configured to store instructions, when executed by at least one processor, at least a portion of the instruction may direct the at least one processor to implement the operations as described above.

The possible beneficial effects of the embodiments of the present disclosure may include but not limited to the following. (1) The accuracy of a blood pressure algorithm can be improved by reasonably using a plurality of calibration data. (2) A blood pressure state of a subject can be obtained accurately and intuitively through a 2D density map and a 3D density map. (3) Taking measurement results of a plurality of subjects as a reference, there is no need to collect many samples from a same subject.

It should be noted that different embodiments may have different beneficial effects. In different embodiments, the possible beneficial effects may include any combination of one or more of the above, or any other possible beneficial effects that may be obtained.

Some embodiments of the present disclosure and/or some other embodiments are described above. Different modifications may also be made in the present disclosure according to above content. The subject matter disclosed in the present disclosure can be implemented in different forms and embodiments, and the present disclosure can be applied to a large number of applications. All applications, modifications and changes claimed in the following claims belong to the scope of the present disclosure.

Moreover, certain terminology has been used to describe embodiments of the present disclosure. As “one embodiment”, “one embodiment”, and/or “some embodiments” means a particular feature, structure or features associated with the present disclosure at least one embodiment. For example, the terms “one embodiment”, “an embodiment”, and/or “some embodiments” mean that a particular feature, structure, or characteristic described in connection with the embodiment is included in at least one embodiment of the present disclosure. Therefore, it is emphasized and should be appreciated that two or more references to “an embodiment” or “one embodiment” or “an alternative embodiment” in various portions of this specification are not necessarily all referring to the same embodiment. In addition, some features, structures, or features in the present disclosure of one or more embodiments may be appropriately combined.

Those skilled in the art will appreciate that there may be a variety of variations and improvements in the contents disclosed herein. For example, the different system components described above are implemented by hardware devices, but may also be implemented only by software solutions. For example, a system may be installed on an existing server. Further, the location information disclosed herein may be provided through a firmware, a combination of firmware/software, a combination of firmware/hardware, or a combination of hardware/firmware/software.

All software or some of them may sometimes communicate via the network, such as the Internet or other communication networks. Such communication can load software from one computer device or processor to another computer device or processor. For example, a hardware platform loaded from a management server or host computer of a system to a computer environment, or other computer environment for realizing the system, or a system with similar functions related to providing information required to determine the target structure parameters. Therefore, another medium that can transmit software elements can also be used as a physical connection between local devices. For example, light waves, electric waves, electromagnetic waves, etc., spread through cables, optical cables or air. The physical media used for carrier waves, such as cables, wireless connections, or optical cables, can also be considered as media carrying software. Unless the usage herein limits the tangible “storage” medium, other terms that refer to the computer or machine “readable medium” all refer to the medium that participates in the process of executing any instructions by the processor.

Computer program code for carrying out operations for aspects of the present disclosure may be written in any combination of one or more programming languages, including an object oriented programming language such as Java, Scala, Smalltalk, Eiffel, JADE, Emerald, C++, C#, VB.NET, Python or the like, conventional procedural programming languages, such as the “C” programming language, Visual Basic, Fortran 2003, Perl, COBOL 2002, PHP, ABAP, dynamic programming languages such as Python, Ruby, and Groovy, or other programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider) or in a cloud computing environment or offered as a service such as a Software as a Service (SaaS).

Furthermore, the recited order of processing elements or sequences, or the use of numbers, letters, or other designations thereof, are not intended to limit the claimed processes and methods to any order except as may be specified in the claims. Although the above disclosure discusses through various examples what is currently considered to be a variety of useful embodiments of the disclosure, it is to be understood that such detail is solely for that purpose, and that the appended claims are not limited to the disclosed embodiments, but, on the contrary, are intended to cover modifications and equivalent arrangements that are within the spirit and scope of the disclosed embodiments. For example, although the implementation of various components described above may be embodied in a hardware device, it may also be implemented as a software only solution, e.g., an installation on an existing server or mobile device.

Similarly, it should be appreciated that in the foregoing description of embodiments of the present disclosure, various features are sometimes grouped together in a single embodiment, figure, or description thereof for the purpose of streamlining the disclosure aiding in the understanding of one or more of the various embodiments. However, this disclosure does not mean that the present disclosure object requires more features than the features mentioned in the claims. Rather, claimed subject matter may lie in less than all features of a single foregoing disclosed embodiment.

In some embodiments, the numbers expressing quantities, properties, and so forth, used to describe and claim certain embodiments of the application are to be understood as being modified in some instances by the term “about”, “approximate”, or “substantially”. For example, “about”, “approximate”, or “substantially” may indicate ±20% variation of the value it describes, unless otherwise stated. Accordingly, in some embodiments, the numerical parameters set forth in the description and attached claims are approximations that may vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable.

Each of the patents, patent applications, publications of patent applications, and other material, such as articles, books, specifications, publications, documents, and/or the like, referenced herein is hereby incorporated herein by this reference in its entirety for all purposes. In addition to the application history documents that are inconsistent or conflicting with the contents of the present disclosure, the documents that may limit the widest range of the claim of the present disclosure (currently or later attached to this application) are excluded from the present disclosure. It should be noted that if the description, definition, and/or terms used in the appended application of the present disclosure is inconsistent or conflicting with the content described in the present disclosure, the use of the description, definition and/or terms of the present disclosure shall prevail.

At last, it should be understood that the embodiments described in the present disclosure are merely illustrative of the principles of the embodiments of the present disclosure. Other modifications that may be employed may be within the scope of the present disclosure. Thus, by way of example, but not of limitation, alternative configurations of the embodiments of the present disclosure may be utilized in accordance with the teachings herein. Accordingly, embodiments of the present disclosure are not limited to that precisely as shown and described.

1. A method for blood pressure calibration selection, which is implemented by a computer device including at least one processor and at least one storage device, comprising:
inputting a sample set, the sample set including data files of a plurality of subjects, the data file of each subject among the plurality of subjects including a plurality of sample photoplethysmography (PPG) waveforms and corresponding blood pressure;
obtaining calibration data of the each subject in the sample set, the calibration data at least including first calibration data and second calibration data in different blood pressure states;
selecting at least one feature parameter of the plurality of sample PPG waveforms;
obtaining a value distribution of a feature parameter among the at least one feature parameter in the sample set based on a plurality of values of the feature parameter in the first calibration data and the second calibration data;
obtaining a comparison result by comparing the feature parameter of a PPG waveform to be detected with the corresponding value distribution; and
determining calibration data corresponding to the PPG waveform to be detected based on the comparison result.

2. The method of claim 1, wherein the first calibration data includes data in a normal blood pressure state, the first calibration data being recorded as low calibration data; and
the second calibration data includes data in a high blood pressure state, the second calibration data being recorded as high calibration data.

3. The method of claim 2, wherein the low calibration data is obtained based on a first process, the first process including:
determining a minimum value of systolic blood pressure of the each subject in the sample set; d
determining data corresponding to the minimum value as the low calibration data.

4. The method of claim 3, wherein the high calibration data is obtained based on a second process, the second process including:
determining data indicating that a difference between systolic blood pressure of the each subject and the minimum value of the systolic blood pressure of the each subject in the sample set is greater than a threshold A and the systolic blood pressure of the each subject is greater than a threshold B; and
determining the data as the high calibration data.

5. The method of claim 4, wherein the threshold A is 20 millimeters of mercury (mmHg), and the threshold value B is 130 mmHg.

6. The method of claim 1, wherein the feature parameter among the at least one feature parameter is determined based on at least one of an original waveform, a first-order derivative waveform, a second-order derivative waveform, a third-order derivative waveform, or a fourth-order derivative waveform of the sample PPG waveform.

7. The method of claim 1, wherein the feature parameter among the at least one feature parameter includes at least one of time amount, area amount, or amplitude amount.

8. The method of claim 1, wherein the obtaining a value distribution of a feature parameter among the at least one feature parameter in the sample set based on a plurality of values of the feature parameter in the first calibration data and the second calibration data includes:
drawing a two-dimensional (2D) density map and/or a three-dimensional (3D) density map for the feature parameter based on the plurality of values of the feature parameter in the first calibration data and the second calibration data.

9. The method of claim 8, wherein the drawing a 2D density map includes:
establishing an XY coordinate system;
obtaining a plurality of discrete points, each of the plurality of discrete points being obtained by setting a value of the feature parameter in the first calibration data corresponding to the each subject as an X-axis coordinate and setting a value of the feature parameter in the second calibration data corresponding to the each subject as a Y-axis coordinate; and
obtaining the 2D density map based on a density distribution of the plurality of discrete points.

10. The method of claim 8, wherein the drawing the 3D density map includes:
generating a set of correct label data and a set of error label data based on a value of the feature parameter in the first calibration data corresponding to the each subject, a value of the feature parameter in the second calibration data corresponding to the each subject, and a value of the feature parameter in a sample PPG waveform of the each subject other than the calibration data.

11. The method of claim 10, wherein the comparing the feature parameter of a PPG waveform to be detected with the corresponding value distribution includes comparing the feature parameter of the PPG waveform to be detected with the 2D density map and/or the 3D density map, including:
generating coordinates of at least two points by combining a value of the feature parameter in the PPG waveform to be detected with the values of the feature parameter in the calibration data; and
obtaining a relationship between the at least two points and a maximum density point in the 2D density map and/or the 3D density map.

12. The method of claim 11, further comprising:
determining a point in the at least two points that is closer to the maximum density point in the 2D density map and/or the 3D density map; and
designating calibration data corresponding to the point as the calibration data corresponding to the PPG waveform to be detected.

13. The method of claim 10, wherein the comparing the feature parameter of a PPG waveform to be detected with the corresponding value distribution includes comparing the feature parameter of the PPG waveform to be detected with the 2D density map and/or the 3D density map, including:
generating coordinates of at least two points by combining a value of the feature parameter in the PPG waveform to be detected with the values of the feature parameter in the calibration data; and
obtaining a distance between each of the at least two points and a point obtained from calibration data related to the PPG waveform to be detected.

14. The method of claim 13, further comprising:
determining a point in the at least two points that is closer to the point obtained from the calibration data related to the PPG waveform to be detected; and
designating calibration data corresponding to the point as the calibration data corresponding to the PPG waveform to be detected.

15. The method of claim 14, wherein an X-axis coordinate and a Y-axis coordinate of the point obtained from the calibration data related to the PPG waveform to be detected are the values of the feature parameter in the calibration data.

16. A modeling method of a method for blood pressure calibration selection, comprising:
inputting a sample set, the sample set including data files of a plurality of subjects, the data file of each subject of the plurality of subjects including a plurality of sample photoplethysmography (PPG) waveforms and corresponding blood pressure;
allocating the sample set into a set of training data and a set of test data;
obtaining calibration data of the set of test data, recording the calibration data of the set of test data as test calibration data, selecting one of the data in the set of test data other than the test calibration data as the test data, determining data with a minimum difference between systolic blood pressure in the test calibration data and systolic blood pressure corresponding to the test data as calibration result data of the test data;
training an initial model based on an input of a sample PPG waveform in the set of training data and an output of corresponding calibration data; and
obtaining an output of a trained model by inputting a sample PPG waveform in the test data, obtaining a comparison result by comparing whether the output of the trained model is consistent with the calibration data, and determining accuracy of the trained model based on the comparison result.

17. The method of claim 16, comprising:
obtaining calibrated data of the set of training data;
recording the calibrated data of the set of training data as training calibration data;
drawing a two-dimensional (2D) density map for at least one feature parameter in the sample PPG waveform based on the training calibration data; and
obtaining a first set of output results by comparing the at least one feature parameter of the test data with the corresponding 2D density map.

18. The method of claim 17, further comprising:
drawing a three-dimensional (3D) density map for the at least one feature parameter in the sample PPG waveform based on the training calibration data; and
obtaining a second set of output results by comparing the at least one feature parameter of the test data with the corresponding 3D density map.

19. The method of claim 18, further comprising:
obtaining a final set of final outputs by processing the first set of output results and the second set of output results according to a collective voting algorithm.

20. (canceled)

21. A device for blood pressure calibration selection, wherein the device comprises at least one processor and at least one memory;
the at least one memory configured to store instructions; and
the at least one processor configured to execute at least a portion of the instructions to implement operations including:
inputting a sample set, the sample set including data files of a plurality of subjects, the data file of each subject among the plurality of subjects including a plurality of sample photoplethysmography (PPG) waveforms and corresponding blood pressure;
obtaining calibration data of the each subject in the sample set, the calibration data at least including first calibration data and second calibration data in different blood pressure states;
selecting at least one feature parameter of the plurality of sample PPG waveforms;
obtaining a value distribution of a feature parameter among the at least one feature parameter in the sample set based on a plurality of values of the feature parameter in the first calibration data and the second calibration data;
obtaining a comparison result by comparing the feature parameter of a PPG waveform to be detected with the corresponding value distribution; and
determining calibration data corresponding to the PPG waveform to be detected based on the comparison result.

22. (canceled)
